(19) United States
(12) Patent Application Publication
(10) Pub. No.: US 2026/0306132 A1
(43) Pub. Date: Oct. 8, 2026

(54) Surgical Tool With Proximally Mounted Camera For Use With Cannulas

(71) Applicant: Viseon, Inc., Irvine, CA (US)

(72) Inventors: Todd D. McIntyre, Irvine, CA (US); Peter G. Davis, Irvine, CA (US)

(73) Assignee: Viseon, Inc., Irvine, CA (US)

(21) Appl. No.: 19/640,191

(22) Filed: Apr. 6, 2026

Related U.S. Application Data
(60) Provisional application No. 63/784,706, filed on Apr. 7, 2025.

(51) Int. Cl. A61B 17/34; A61B 1/00; A61B 1/05; A61B 1/313; A61B 17/00

(52) U.S. Cl. CPC A61B 17/3421; A61B 1/00097; A61B 1/05; A61B 1/313; A61B 2017/00026; A61B 2217/005

(57) ABSTRACT

An aspirator having an aspirator and nerve monitoring system, including sensors configured for placement on the body of the patient, in locations at which the sensors can detect physiological signals induced by stimulation signals transmitted through the aspirator tube. The nerve monitoring system includes a control system for initiating the stimulation signal and receiving signal from the sensors, and provide output to the surgeon which correspond to the signal from the sensors which are indicative of the proximity of the aspirator tube tip to the nerve. A surgeon can use the monitoring system while the aspirator tube tip is in the brain of the patient to send stimulation signals into the brain and manipulate the aspirator tube to avoid aspiration in such close proximity to the nerve that aspiration might damage the nerve.

This application claims priority to U.S. Provisional Application 63/784,706, filed Apr. 7, 2025, pending.

### FIELD OF THE INVENTIONS

The inventions described below relate to the field of minimally invasive brain surgery.

### BACKGROUND

In our prior patent publication, McIntyre and Davis, Image Overlay Method for Nerve Monitoring Systems, U.S. Publication 2025/0148599 (May 5, 2025) we disclosed systems and methods for identification and visualization of nerves proximate a surgical site within the brain during brain surgery. One embodiment of that system included an aspirator fitted with a camera assembly, at a position at the proximal end of aspirator tube such that it resides outside the body when the aspirator tube tip is disposed in the surgical workspace, providing images of tissue in the workspace while a surgeon manipulates the tool within the surgical workspace. The system also includes a nerve monitoring system which includes an electrode on the distal end of the aspirator tube and response sensors suitable for mounting on the patient's skin, in areas expected to provide signals indicative of stimulation of the nerve. The nerve monitoring system is interoperable with the fluoroscopic imaging system to determine the location of the tool tip corresponding to a signal from the response sensors, and present, on a fluoroscopic display of the patient's brain, a screen element in a corresponding location in the fluoroscopic images.

### SUMMARY

The systems and methods describe below provide for aspiration of masses within the brain, such as tumors or blood masses, while providing a mechanism to help a surgeon avoid damaging nerves near the mass. The system includes an aspirator having an aspirator tube with a camera assembly mounted on the proximal end. of the aspirator tube, components of a nerve monitoring system incorporated into the aspirator tube, and sensors configured for placement on the body of the patient, in locations at which the sensors can detect physiological signals induced by stimulation signals transmitted through the aspirator tube. The sensors can be configured for placement over regions of the brain which may react to stimulation of an associated nerve, such as the occipital region of the scalp which overlies the visual cortex, which can react and produce detectable physiological signals in response to stimulation of the optic nerve. The nerve monitoring system also includes a control system for initiating the stimulation signal and receiving signal from the sensors, and provide output to the surgeon which correspond to the signal from the sensors which are indicative of the proximity of the aspirator tube tip to the nerve. A surgeon can use the monitoring system while the aspirator tube tip is in the brain of the patient to send stimulation signals into the brain and manipulate the aspirator tube to avoid aspiration in such close proximity to the nerve that aspiration might damage the nerve.

### BRIEF DESCRIPTION OF THE DRAWINGS

FIG. 1 illustrates a system comprising a surgical tool, such as a aspirator, with a proximally mounted camera, in a cannula, with a nerve monitoring stimulation source.

FIG. 2 depicts a system operable to detect proximity of the distal tip of the tool to nerve within the brain.

### DETAILED DESCRIPTION OF THE INVENTIONS

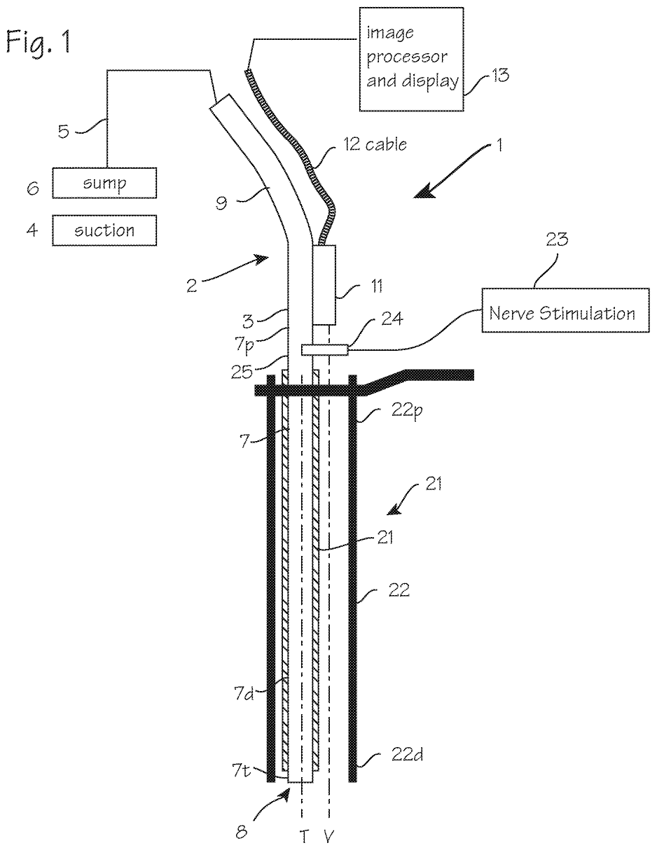

FIG. 1 illustrates surgical system 1 including an aspirator system 2 comprising an aspirator tube 3 and a vacuum source 4 connected to the aspirator tube 3 through a suction line 5 and a sump 6 for collection of any tissue collected through the aspirator tube 3. The aspirator tube 3 comprises an insertion portion 7 which typically consists of a slender tube with a distal portion 7d configured for insertion into a surgical workspace and a proximal portion 7p which typically remains outside the surgical workspace (outside the body of a patient) during use. A suction port 8 is located at or very near the distal tip 7t of the distal portion 7d. The distal tip may be straight cut or beveled. Preferably, the aspirator tube also comprises a connection portion 9 extending proximally from the insertion portion, and this connection portion may be offset from the straight insertion portion 7 at an angle so that the aspirator tube may be held at a point offset from the straight insertion portion 7, so that the surgical workspace is not obscured by the surgeon's hand or other manipulator during use. The straight insertion portion 7 of the aspirator tube is characterized by a longitudinal axis T, and is preferably configured for insertion into a surgical workspace through a cannula (straight enough and long enough to reach a surgical workspace at the distal end of a cannula with which it is to be used).

The surgical system includes a camera assembly 11 mounted on the proximal portion 7p of the straight insertion portion 7. The camera assembly is connected through a video cable 12 to an image processor and video display 13. The camera assembly is characterized by a viewing axis V, which is aligned substantially parallel to the axis T of the straight insertion portion 7 of the aspirator tube. Alternatively, the camera assembly may be mounted proximally to the straight insertion portion 7, on the offset connection portion 9, in which case the camera assembly may include prisms or mirrors that may be located on the proximal portion 7p so as to locate the viewing axis parallel to the axis T of the straight insertion portion 7. The distal most component, which may be an optical surface (a lens, filter, prism or mirror), is located proximally relative to the straight insertion portion 7, well-spaced from the distal portion 7d, so that it does not block the view of the surgeon into the surgical space around the distal tip 7t.

The surgical system preferably includes a cannula 21 with a cannula tube 22 configured for insertion into the body of a patient, such that the distal end 22d is located within the body with the open distal end of the tube near a surgical workspace and the open proximal end 22p. The aspirator 3 is configured such that, with the distal portion 7d disposed within the cannula, and the distal tip 7t proximate the distal opening of the cannula tube and/or extending distally from the distal end 22d and/or into the surgical workspace, the proximal portion 7p of the insertion portion 7 extends proximally out of the cannula tube and the camera assembly, including the distal most component, is disposed proximally of the open proximal end 22p of the cannula tube 22.

The aspirator and camera can be used with bladed retractors, or without retractors, employing the methods described above. While the invention has been illustrated within a system for aspiration, other surgical tools may be fitted with a proximally mounted camera and used as described above.

FIG. 1 also illustrates a nerve monitoring stimulation source. The aspirator tube, or a portion of the aspirator tube, comprises an electrical conductor establishing a conductive pathway from the proximal portion 7p to the distal portion 7d and, preferably, the distal tip 7t of the aspirator tube 7. The aspirator tube preferably comprises a metal tube, preferably stainless steel, and the entire insertion portion comprises the conductive pathway. An insulative covering 21 (a sleeve or coating) is disposed over the aspirator tube, leaving a proximal portion uncovered, and leaving the distal portion or distal tip uncovered. A nerve stimulation source 23 is operable to pass nerve stimulation signals through a connector 24 to the bare portion 25 of the proximal portion 7p, and hence through the aspirator tube 7 to the distal portion 7d and, preferably, the distal tip 7t of the aspirator tube 7. The distal tip 7t acts as an electrode, configured for passing the electrical nerve stimulation signals into the body, though a discrete electrode mounted on the distal tip may be used. Using this nerve stimulation system, a surgeon can operate the nerve stimulation source 23 to pass signals into tissue around the distal portion 7d and/or distal tip 7t. A sensor, such as a surface electrode, may be placed on the patient in an area innervated by and responsive to stimulation of a nerve passing through tissue proximate the distal tip 7t when the tip is inserted into a surgical workspace, or a location of the brain responsive to stimulation of a nerve passing through tissue proximate the distal tip 7t when the tip is inserted into a surgical workspace. The sensor are operable to detect a physiological response, such as electrical signal generated by the brain or motion signals indicative of motion areas of the body enervated by the nerve. Other sensors may be used, for example EEG sensors or intracranial leads operable to detect brain activity that is typically a response to stimulation of a nerve passing through tissue in the vicinity of the distal portion 7d and/or distal tip 7t.

For example, when inserting the aspirator tube tip into the brain proximate the optic nerve (CN II) (which is difficult to see when aspirating nearby), an optic nerve response system using electrodes on the head can detect stimulation of the optic nerve, similar to electroretinography. When inserting the aspirator tube tip into the brain proximate the auditory nerve (which is difficult to see when aspirating nearby), an auditory brainstem response monitoring system using an electrode on the scalp proximate the temporal lobe and auditory cortex patient, or placed in the ear can detect stimulation of the auditory nerve or auditory cortex. Electrocochleography, direct monitoring of the eighth cranial nerve, and direct monitoring of the auditory nerve (the vestibulocochlear nerve, CN VIII) can be used as well, to detect stimulation of the auditory nerve by the nerve stimulation. The auditory nerve and many other nerves may be monitored for contact with or close proximity to the distal tip 7t. Thus, the physiological response may be generated by the patient's body at a point remote from the distal tip and portion of the nerve stimulated by the stimulation. Other nerves which may affected by aspiration of a tissue mass within the brain, and thus benefit from the protect provided by the system and method, include the cranial nerves (the olfactory nerve (CN I), the oculomotor nerve (CN III), the trochlear nerve (CN IV), the trigeminal nerve (CN V), the abducens nerve (CN VI), the facial nerve (CN VII), the), the glossopharyngeal nerve (CN IX), the vagus nerve (CN X), the accessory nerve (CN XI), and the hypoglossal nerve (CN XII).

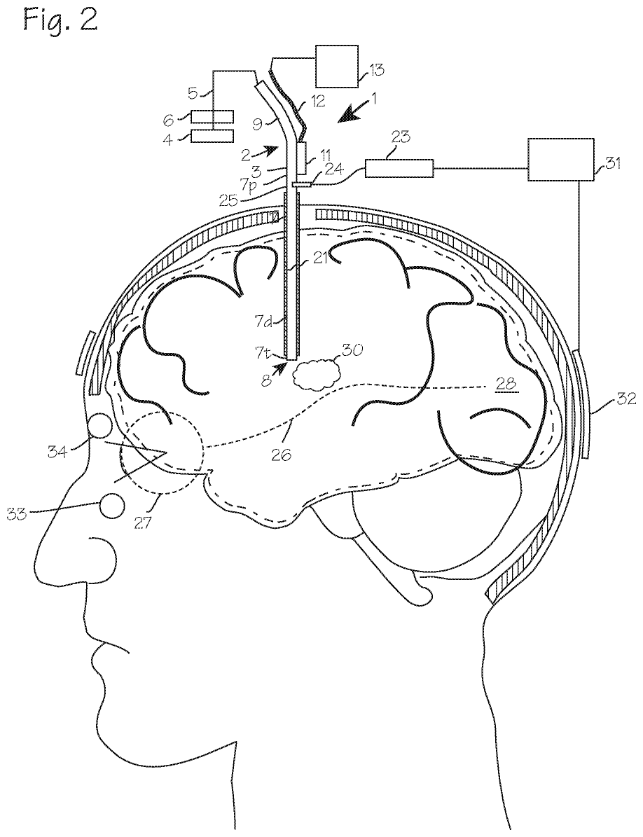

FIG. 2 depicts a monitoring system operable to detect proximity of the distal tip 7t of the tool to the optical tool. This FIG. 2 depicts the optic nerve 26 in its course from the eye 27 to the visual cortex 28. A tumor or blood mass 30 is depicted in close proximity to the optic nerve. To detect stimulation of the optic nerve, a system similar to an electroretinography system can be used. The system includes the nerve stimulation source 23, a control system 31, a surface electrode which may be located on the occipital region of the scalp proximate the visual cortex (electrode 32) or under the eye or on the temple (electrodes 33 and/or 34), and optionally, a reference electrode 35 on the forehead and a ground electrode 36 elsewhere on the patient. The control system is operable to initiate transmission of an electrical signal from the nerve stimulation source 23 and detect electrical signals emitted by the visual cortex or retina in response to that stimulation.

The system may be configured to output a signal to the surgeon which varies with the physiological signal detected by the sensor. The signal may be audible or visual and may vary according to the physiological signal detected by the sensor. The surgeon can then manipulate the aspirator tube while operating the monitoring system and observe the output of the monitoring system and operate the aspirator tool when the signal indicates that the aspirator tube tip is safely spaced from the nerve, and avoid operation of the aspirator tube when the signal indicates that the aspirator tube tip is very close to the nerve. The sensor may detect no signal (no signal above normal activity) responsive to the stimulation signal, which would indicate no proximity of the aspirator tube tip to the nerve, a slight signal above normal activity which may be taken to indicate safe spacing of the tip from the nerve, or a large signal above normal activity which may be taken as an indication that the aspirator tube tip it close to the nerve (and that operation of the aspirator in such proximity to the nerve might damage the nerve.)

The control system may be configured to output a signal to the surgeon (an audible or visual indication) that indicates that the system has detected a response (a physiological, electrical response, for example) to the stimulation signal, which may be detectable electrical activity of a portion of the brain (for example, the visual cortex in the occipital lobe or auditory cortex in the temporal lobe). The control system will preferably be operable to output a first single indicative of detection of a first level of activity which is consistent with the location of the distal tip a safe distance from the nerve, and a second signal indicative of detection of a second level of activity which is consistent with a location of the distal tip undesirably close to the nerve. The first and second level of activity may vary depending on the area of the brain being treated, the level of electrical stimulation applied, the nerve stimulated, and the location of the electrodes, and can be adjusted as experience dictates. The first and second level may be predetermined and pre-programmed into the control system, or set by the surgeon and input into the control system prior to use.

The aspiration tube itself may be used to conduct stimulation signals, but other arrangements may be used. For example, a wire may be trapped between the aspirator tube and sleeve 21, exposed beyond the distal end of the sleeve, and may be used. The wire may be resiliently deformable, secured to the distal tip, such that it may deform resiliently into an arc extending radially from the tube, and operated by pushing the proximal end of the wire distally.

In use, a surgeon will install the cannula in a surgical opening or anatomical opening in the patient, insert the insertion portion 7 of the aspirator tube 3 into the cannula so that the distal tip 7t and suction port 8 are within the surgical workspace in the brain. The surgeon will operate the camera assembly, image processor and display to obtain video images of the workspace near the distal tip and display those images on the display. The surgeon may then operate the aspirator to collect tissue from the workspace, using the video images from the camera to guide the distal tip 7t within the surgical workspace and collect tissue. As the surgeon is manipulating the tool to place the distal tip within a mass tissue to be aspirated (typically a tumor or blood mass within the brain), the surgeon will operate the control system (either manually or automatically through operation of the control system) to transmit an electrical stimulation signal through the aspirator tube and into tissue proximate the distal tip of the aspirator tube. The surgeon will monitor the control system for output of signals that indicate that the system has detected a response to the stimulation signal. In response to a signal that indicates that the system has detected a response to the stimulation signal at the second level mentioned above (indicating undesirable proximity of the distal tip of the aspirator tube to the nerve, the surgeon may stop the operation of the aspirator and re-position the distal tip and continue operation of the control system and monitor the control system for output of signals that indicate that the system has detected a response to the stimulation signal at the first level mentioned above (indicating acceptable proximity of the distal tip of the aspirator tube to the nerve). Note that there is no need to contact or impinge upon the nerve for the stimulation signal to affect the nerve and thereby induce a detectable physiological response.

As an alternative, the electrical stimulation signal may be passed into the surgical site through an insulated wire along the aspirator tube (on the outer surface, inner surface, or within the tube wall) and an electrode at the distal tip of the aspirator tube.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised.

We claim:

1. A surgical system for removal of tissue from a surgical workspace with the body of a patient, said system comprising:
an aspirator system comprising an aspirator tube, said aspirator tube having a proximal end, a distal end and a distal tip, with a lumen extending from the proximal end to the distal end, a suction port at the distal end, said distal end configured for insertion into the surgical workspace, and a vacuum source connected to the aspirator tube;
a camera assembly mounted on the proximal end of the aspirator tube, said camera assembly having a viewing axis oriented substantially parallel to the long axis of the distal end of the aspirator tube; wherein
the aspirator tube comprises an electrical conductor establishing a conductive pathway between the proximal end and the distal end;
an insulative covering disposed over at least a portion of the aspirator tube between the proximal end and the distal end of the aspirator tube, leaving a conductive region of the proximal end of the aspirator tube and a conductive region of the distal end uncovered; and
a nerve stimulation source electrically coupled to the conductive region of the proximal end of the aspirator tube and operable to deliver an electrical stimulation signal through the conductive pathway to the conductive region of the distal end of the aspirator tube for application to tissue adjacent the distal tip.

2. The surgical system of claim 1, further comprising:
a monitoring system, said monitoring system comprising:
a sensor configured for attachment to the patient at a region responsive to stimulation of a nerve passing through tissue proximate the distal tip when the distal tip is inserted into a surgical workspace, said sensor operable to detect a biological response to stimulation of said nerve;
a control system configured to:
operate the nerve stimulation source to deliver the electrical stimulation signals;
receive signals picked up by the sensor;
output a signal to the surgeon (an audible or visual indication) in response to receiving the signals from the sensor indicative of physiological response to the stimulation signal.

3. The surgical system of claim 2, wherein the control system is further configured to
output a first signal indicative of detection of a first level of activity which is consistent with the location of the distal tip a safe distance from the nerve, and a second signal indicative of detection of a second level of activity which is consistent with a location of the distal tip undesirably close to the nerve.

4. The surgical system of claim 2, wherein:
the control system is further configured to output a signal to the surgeon which varies with the biological signal detected by the sensor.

5. The surgical system of claim 1, wherein:
the distal end of the aspirator tube is characterized by a distal tip and the uncovered conductive region of the distal end is limited to the distal tip.

6. The surgical system of claim 2, wherein:
the sensor comprises a surface electrode configured for placement on the head of the patient.

7. The surgical system of claim 2, wherein:
the sensor comprises a surface electrode configured for placement on an occipital region of the scalp of the patient.

8. The surgical system of claim 2, wherein:
the sensor comprises an electrode configured for placement on an ear of the patient.

9. The surgical system of claim 2, wherein:
the control system is configured to generate an output indicative of proximity between the distal tip and a nerve, based on the detected physiological signal.

10. A surgical system for removal of tissue from a surgical workspace with the body of a patient, said system comprising:
an aspirator tube;
a vacuum source;
a wire extending along the aspirator tube and electrically insulated from at least a portion of the aspirator tube;
an electrode at a distal end of the wire positioned adjacent a distal tip of the aspirator tube; and
a nerve stimulation source electrically coupled to the wire to deliver stimulation signals to tissue adjacent the distal tip.

11. A method of removing tissue from a surgical workspace within the body of a patient, said method comprising:
providing an aspirator system comprising
an aspirator tube, said aspirator tube having a proximal end and a distal end, with a lumen extending from the proximal end to the distal end, a suction port at the distal end, said distal end configured for insertion into the surgical workspace, and a vacuum source connected to the aspirator tube;
a camera assembly mounted on the proximal end of the aspirator tube, said camera assembly having a viewing axis oriented substantially parallel to the long axis of the distal end of the aspirator tube;
wherein at least a portion of the aspirator tube comprises an electrical conductor establishing a conductive pathway between the proximal end and the distal end;
an insulative covering disposed over at least a portion of the surgical tool between the proximal end and the distal end of the aspirator tube, leaving a conductive region of the proximal end of the aspirator tube and a conductive region of the distal end uncovered; and
a nerve stimulation source electrically coupled to the conductive region of the proximal end of the aspirator tube and operable to deliver an electrical stimulation signal through the conductive pathway to the conductive region of the distal end of the aspirator tube for application to tissue adjacent the distal tip;
and providing monitoring system comprising:
a sensor configured for attachment to the patient at a region responsive to stimulation of a nerve passing through tissue proximate the distal tip when the distal tip is inserted into a surgical workspace, said sensor operable to detect a biological response to stimulation of said nerve;
a control system configured to:
operate the nerve stimulation source to deliver the electrical stimulation signals;
receive signals picked up by the sensor;
output a signal to the surgeon (an audible or visual indication) in response to receiving the signals from the sensor indicative of biological response to the stimulation signal;
placing the sensor on the patient at a region of the patient responsive to stimulation of a nerve;
inserting the distal end of the aspirator tube into the body of the patient;
operating the vacuum source to apply suction to the tissue to aspirate tissue from the body;
operating the control system to deliver an electrical stimulation signal to the tissue, receive signals indicative of biological response to the stimulation signal detected by the sensor, and output a signal to the surgeon in response to receiving signals from the sensor indicative of physiological response to the stimulation signal.

12. The method of claim 11, wherein the nerve is an optic nerve, and the sensor is place on an occipital area of the scalp of the patient.

13. The method of claim 11, wherein the nerve is an optic nerve, and the sensor is placed on the face of the patient.

14. The method of claim 11, wherein the nerve is an auditory nerve, and the sensor is placed on or in an ear of the patient.

15. The method of claim 11, wherein the nerve is an auditory nerve, and the sensor is placed proximate the temporal lobe and auditory cortex patient.
